(19) United States
(12) Patent Application Publication
(10) Pub. No.: US 2009/0306746 A1
(43) Pub. Date: Dec. 10, 2009

(54) SYSTEM AND METHOD FOR PROGRAMMING AN IMPLANTABLE NEUROSTIMULATOR

(76) Inventor: Brian BLISCHAK, Allen, TX (US)

Correspondence Address: ST. JUDE MEDICAL NEUROMODULATION DIVISION, 6901 PRESTON ROAD, PLANO, TX, 75024, US

(21) Appl. No.: 11/671,676

(22) Filed: Feb. 6, 2007

Related U.S. Application Data
(60) Provisional application No. 60/771,315, filed on Feb. 7, 2006.

(51) Int. Cl. A61N 1/08

(52) U.S. Cl. 607/59

(57) ABSTRACT

According to one embodiment, a computer-implemented system is provided for programming an implantable neurostimulator. A memory module stores relative positioning data representing determined relative positioning in at least two dimensions of an electrode in a first implanted neurostimulation lead relative to an electrode in a second implanted neurostimulation lead or to neural structures. A processing module coupled to the memory module accesses the relative positioning data stored in the memory module, determines one or more stimulation characteristics according to the accessed relative positioning data, and communicates the one or more stimulation characteristics determined according to the accessed relative positioning data to an implantable neurostimulator, electrically coupled to the implanted stimulation lead(s), to control operation of the implantable neurostimulator.

### RELATED APPLICATIONS

The present application claims the benefit of Provisional Patent Application Ser. No. 60/771,315, filed Feb. 02, 2006, entitled “SYSTEM AND METHOD FOR PROGRAMMING AN IMPLANTABLE NEUROSTIMULATOR,” which is incorporated herein by reference.

### TECHNICAL FIELD

This application relates generally to therapeutic neurological stimulation and in particular to a system and method for programming an implantable neurostimulator.

### BACKGROUND

Neurological stimulation may be applied to targeted tissue within the body to treat a variety of clinical conditions such as chronic pain. For electrical stimulation, typically, an implanted pulse generator transmits a pulse of electrical energy to one or more implanted neurostimulation leads according to a set of stimulation parameters. Each lead typically has one or more electrodes that are positioned proximate the targeted tissue to be stimulated. These electrodes deliver the electrical energy to the targeted tissue to achieve the desired stimulation.

One form of neurological stimulation is spinal cord stimulation (SCS). Typically, SCS techniques are used to treat chronic pain by stimulating targeted areas of the spinal cord. The stimulation results in a sensation of numbness or tingling in the affected regions of the body, known as “paresthesia.” These techniques may be used to relieve pain in areas of the body by replacing the pain with paresthesia. However, in order to stimulate the spinal cord in such a way as to treat pain in specific parts of the body, the neurostimulation lead or leads must be precisely positioned proximate specific portions of the spinal cord, and the spinal cord must be stimulated using electrical pulses with the proper amplitude, frequency, and pulse width, and using electrodes with the proper polarities relative to one another.

Often multiple neurostimulation leads are utilized in order to achieve the desired stimulation. Due to the imperfect nature of the lead implantation process, these leads are often implanted at different heights along the spinal cord and at different angles with respect to the spinal cord, both medial-laterally and dorsal-ventrally. Once the leads have been implanted, stimulation pulses are utilized to direct the electrical energy through the targeted nerve tissue. The parameters of these pulses are established by a program, usually generated by or with the aid of the implanting physician. The program typically identifies which electrodes will be used to deliver the stimulation pulses, the direction that the pulses will travel between the electrodes based on their relative polarities, and the stimulation parameters (e.g., amplitude, frequency, and pulse width) of the pulses. Such characteristics, or any subset of such characteristics, may be referred to as stimulation characteristics.

Various techniques have been developed to generate such programs. Most of these techniques involve some form of trial and error to test various stimulation characteristics for efficacy. However, due to the number of possible combinations of electrodes, relative polarities, and stimulation parameters, testing all of the possible combinations can be prohibitively time-consuming. In addition, due to the imperfect nature of the lead implantation process, it can be difficult to identify which of the possible combinations provides an optimum or otherwise satisfactory therapeutic result under the circumstances. Accordingly, in order to reduce the amount of time required to identify an optimum or other particular combination, some previous systems make assumptions regarding the relative positioning of electrodes in different implanted leads. For example, some previous systems assume that two implanted leads are: (1) in the same coronal or paracoronal plane (i.e., a plane generally parallel to the spinal cord that divides the torso into anterior and posterior portions); (2) substantially parallel to each other within the plane; (3) a predetermined distance apart from each other within the plane; and (4) inserted the same distance cephalad (i.e., toward the head along the spinal cord) within the plane.

### SUMMARY

Certain embodiments of the present invention may reduce or eliminate certain problems and disadvantages associated with previous techniques for programming an implantable neurostimulator.

According to one embodiment, a computer-implemented system is provided for programming an implantable neurostimulator. A memory module stores relative positioning data representing determined relative positioning in at least two dimensions of an electrode in a first implanted neurostimulation lead relative to an electrode in a second implanted neurostimulation lead. A processing module coupled to the memory module accesses the relative positioning data stored in the memory module, determines one or more stimulation characteristics according to the accessed relative positioning data, and communicates the one or more stimulation characteristics determined according to the accessed relative positioning data to an implantable neurostimulator, electrically coupled to the first and second implanted stimulation leads, to control operation of the implantable neurostimulator.

Particular embodiments of the present invention may provide one or more technical advantages. For example, certain embodiments provide techniques for determining and storing relative positioning information about electrodes in different implanted leads to allow for improved programming of an associated implantable neurostimulator. In certain embodiments, such programming may be at least partially automated based on such determined relative positioning information. Certain embodiments may use such determined relative positioning information in lieu of or as a supplement to assumptions, which are typically incorrect, regarding the relative positioning of electrodes in different implanted leads to reduce the combinations of stimulation characteristics that need to be tested to identify those that yield an optimum or otherwise satisfactory therapeutic result. For example, using such determined relative positioning information, certain embodiments may allow a clinician or automated programmer to more accurately determine the most appropriate electrodes and relative polarities to use to stimulate the targeted nerve tissue. As another example, using such determined relative positioning information, certain embodiments may allow a clinician or automated programmer to more accurately determine the most appropriate stimulation parameters (e.g., amplitude, frequency, and pulse width) to use to stimulate the targeted nerve tissue using these electrodes. As a particular example, certain embodiments may allow a clinician or automated programmer to determine a current amplitude for a current source coupled to an electrode. Certain embodiments may provide all, some, or none of these advantages. Certain embodiments may provide one or more other technical advantages, one or more of which may be readily apparent to those skilled in the art from the figures, descriptions, and claims included herein.

The foregoing has outlined rather broadly certain features and/or technical advantages in order that the detailed description that follows may be better understood. Additional features and/or advantages will be described hereinafter which form the subject of the claims. It should be appreciated by those skilled in the art that the conception and specific embodiment disclosed may be readily utilized as a basis for modifying or designing other structures for carrying out the same purposes. It should also be realized by those skilled in the art that such equivalent constructions do not depart from the spirit and scope of the appended claims. The novel features, both as to organization and method of operation, together with further objects and advantages will be better understood from the following description when considered in connection with the accompanying figures. It is to be expressly understood, however, that each of the figures is provided for the purpose of illustration and description only and is not intended as a definition of the limits of the appended claims.

### BRIEF DESCRIPTION OF THE DRAWINGS

FIG. 1 illustrates an example system for programming an implantable neurostimulator.

FIG. 2 illustrates two example neurostimulation leads implanted adjacent nerve tissue in a patient's spinal cord according to one representative embodiment.

FIGS. 3A-3C illustrate examples of relative positioning of electrodes in two example neurostimulation leads according to one representative embodiment.

FIGS. 4A-4D illustrate example electrode data according to representative embodiments.

FIG. 5 illustrates an example method for use in programming an implanted neurostimulation lead according to one representative embodiment.

### DETAILED DESCRIPTION

According to one embodiment, a computer-implemented system is provided for programming an implantable neurostimulator. A memory module stores relative positioning data representing determined relative positioning in at least two dimensions of an electrode in a first implanted neurostimulation lead relative to an electrode in a second implanted neurostimulation lead. A processing module coupled to the memory module accesses the relative positioning data stored in the memory module, determines one or more stimulation characteristics according to the accessed relative positioning data, and communicates the one or more stimulation characteristics determined according to the accessed relative positioning data to an implantable neurostimulator, electrically coupled to the first and second implanted neurostimulation leads, to control operation of the implantable neurostimulator.

Particular embodiments of the present invention may provide one or more technical advantages. For example, certain embodiments provide techniques for determining and storing relative positioning information about electrodes in different implanted leads to allow for improved programming of an associated implantable neurostimulator. In certain embodiments, such programming may be at least partially automated based on such determined relative positioning information. Certain embodiments may use such determined relative positioning information in lieu of or as a supplement to assumptions, which are typically incorrect, regarding the relative positioning of electrodes in different implanted leads to reduce the combinations of stimulation characteristics that need to be tested to identify those that yield an optimum or otherwise satisfactory therapeutic result. For example, using such determined relative positioning information, certain embodiments may allow a clinician or automated programmer to more accurately determine the most appropriate electrodes and relative polarities to use to stimulate the targeted nerve tissue. As another example, using such determined relative positioning information, certain embodiments may allow a clinician or automated programmer to more accurately determine the most appropriate stimulation parameters (e.g., amplitude, frequency, and pulse width) to use to stimulate the targeted nerve tissue using these electrodes. As a particular example, certain embodiments may allow a clinician or automated programmer to determine a current amplitude for a current source coupled to an electrode. Certain embodiments may provide all, some, or none of these advantages. Certain embodiments may provide one or more other technical advantages, one or more of which may be readily apparent to those skilled in the art from the figures, descriptions, and claims included herein.

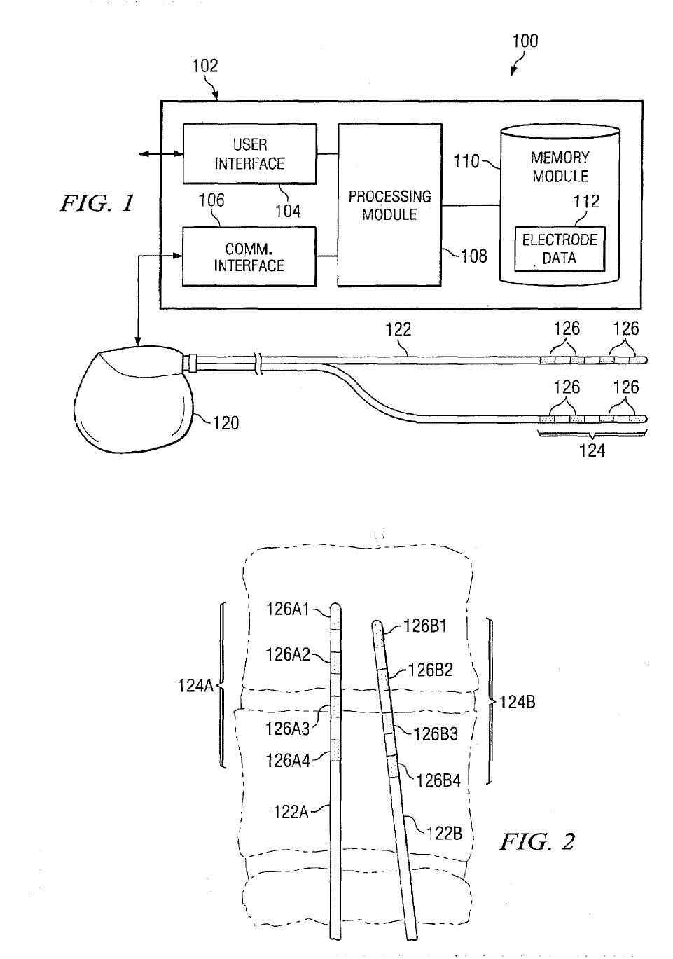

FIG. 1 illustrates an example system 100 for programming an implantable neurostimulator 120. When implanted in a patient, neurostimulator 120 is coupled to two or more implanted neurostimulation leads 122, each having a stimulation portion 124 with one or more electrodes 126. In the embodiment illustrated, a programming module 102 includes a user interface 104, a data interface 106, a processing module 108, and a memory module 110. These components enable programming module 102 to receive information from a user or an associated computer system reflecting relative positioning of electrodes 126 in different implanted neurostimulation leads 122, store that information, use that information to determine appropriate stimulation characteristics for neurostimulator 120, and transmit those determined stimulation characteristics to neurostimulator 120 to control its operation. These components are provided merely as representative examples; the present invention contemplates any components suitable to provide the functionality described herein. In some embodiments, the relative positioning of electrodes 126 is downloaded from programming module 102 and permanently stored in memory (not shown) of neurostimulator 120. If reprogramming is necessary, the stored information can be retrieved by the same or a different programming module 102 to assist the programming process. Other suitable information could be stored in memory of the neurostimulator 120 such as lead types, electrode spacing(s), electrode configurations, etc. Additionally, anatomical structures or locations can be identified relative to the lead(s) and electrode(s).

User interface 104 communicates information to and receives information from a user, such as a clinician, and/or an associated computer system. For example, user interface 104 may be coupled to a keyboard, mouse, touch pad, or other input device to receive information from a user and may be coupled to a display monitor to provide information to the user. As another example, user interface 104 may be coupled to an interface of a computer system associated with the user to receive information directly from and provide information directly to the computer system. User interface 104 may include any suitable software or other logic, in combination with appropriate hardware, to communicate information to and from a user and/or an associated computer system.

Data interface 106 communicates information to and may additionally receive information from neurostimulator 120. In certain embodiments, the information may be communicated wirelessly. For example, data interface 106 may communicate stimulation characteristics to an implanted neurostimulator 120 through the skin using radiofrequency (RF) transmissions. Data interface 106 may include any suitable software or other logic, in combination with appropriate hardware, to communicate information to and from neurostimulator 120.

In certain embodiments, user interface 104 and data interface 106 may be combined into a single component. In certain embodiments, user interface 104, data interface 106, or both may be distributed across multiple components.

Processing module 108 controls the operation of other components within programming module 102 according to information received from user interface 104, data interface 106, and memory module 110. For example, processing module 108 may receive information from user interface 104, store some or all of the information in memory module 110, and transmit some or all of the information to data interface 106 for transmission to neurostimulator 120. In particular, processing module 108 determines, based on determined relative positioning information for electrodes 126 of different implanted neurostimulation leads 122 received through user interface 104, stimulation characteristics to be transmitted to neurostimulator 120 to control its operation. Processing module 108 may include any suitable software or other logic, in combination with appropriate hardware, to control and process information. For example, processing module 108 may be a microcontroller or other programmable logic device or combination of devices.

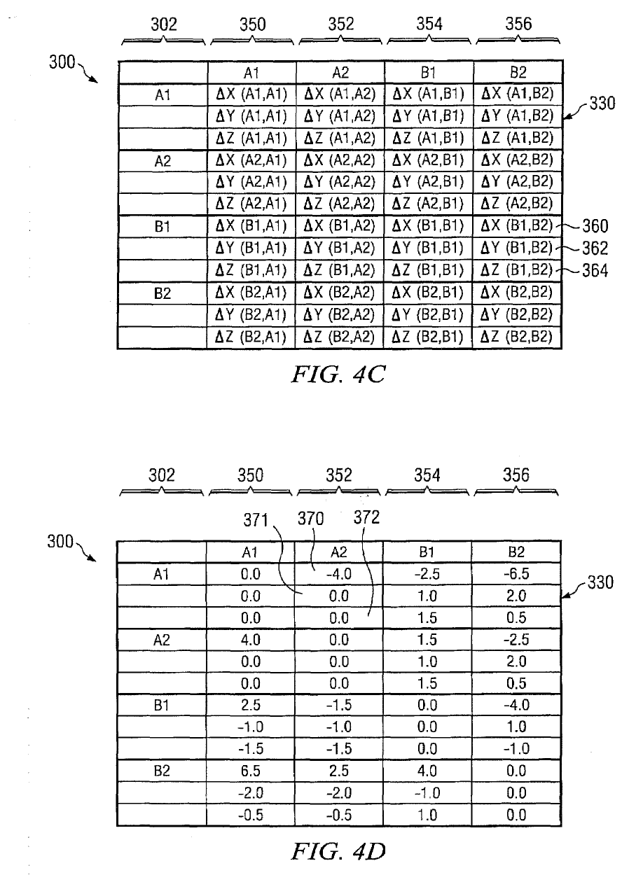

Memory module 110 stores, either permanently or temporarily, information for processing by processing module 108 and communication by user interface 104 and data interface 106. Under the control of processing module 108, memory module 110 stores electrode positioning data 112, described below, reflecting the relative positioning of electrodes 126 (e.g., relative to each other and/or relative to neural tissue of the spinal cord or brain tissue as examples) in different implanted neurostimulation leads 122. In some embodiments (for reprogramming), the information is retrieved from memory of neurostimulator 120 using communication interface 106 and the information is temporarily stored in memory module 110. Memory module 110 may include any volatile or nonvolatile memory suitable for storing such information. For example, memory module 110 may include random access memory (RAM), read only memory (ROM), magnetic storage, optical storage, or any other suitable data storage device or combination of devices. Furthermore, memory module 110 may store information using any suitable structure, format, or technique. In certain embodiments, electrode positioning data 112 may be organized according to the table structure illustrated in FIGS. 4A-4D.

Neurostimulator 120 is an implantable neurostimulation device that, when implanted in a patient, is electrically coupled to electrodes 126 on two or more implanted neurostimulation leads 122. For example, leads 122 may be percutaneous leads and electrodes 126 may be circumferential electrodes. Alternatively, at a given segment of the lead, multiple discrete electrodes 126 can be provided such that each individual electrode 126 only occupies a segment of the circumference of the lead. However, the present invention contemplates any suitable leads 122 and any suitable electrodes 126. In operation, neurostimulator 120, neurostimulation leads 122, and electrodes 126 apply electrical stimulation pulses to targeted nerve tissue within the body, such as targeted nerve tissue in the spinal cord. Neurostimulator 120 controls the stimulation pulses transmitted to electrodes 126 and delivered to the targeted nerve tissue according to programmed stimulation parameters. These stimulation parameters may include, for example, amplitude, frequency, and pulse width. Using programming module 102 or otherwise, a clinician, the patient, or another user of neurostimulator 120 may directly or indirectly input or modify stimulation parameters to specify or modify the nature of the electrical stimulation provided.

In certain embodiments, neurostimulator 120 may be an implantable pulse generator (IPG). For example, neurostimulator 120 may be an IPG manufactured by Advanced Neuromodulation Systems, Inc., such as the Eon® system, or an IPG manufactured by Advanced Bionics Corporation, such as the Precision® system. In preferred embodiments, neurostimulator 120 may include an implantable wireless receiver. In these embodiments, the wireless receiver may be capable of receiving wireless signals from an associated wireless transmitter located external to the patient's body. In certain embodiments, programming module 102 may couple to such a wireless transmitter for transmitting wireless signals to neurostimulator 120.

In operation, programming module 102 receives information representing relative positioning of electrodes 126 of one or more implanted neurostimulation leads 122 and uses that information to determine suitable stimulation characteristics for neurostimulator 120 to control its operation. The relative positioning information may define the position of the electrodes relative to each other and/or relative to anatomic structures. The stimulation characteristics may include which electrodes 126 will be selected to deliver the stimulation pulses (which may change any number of times during a stimulation session), the direction that the stimulation pulses will travel between the selected electrodes 126 based on their relative polarities (which may change any number of times during the stimulation session), and the stimulation parameters (e.g., amplitude, frequency, and pulse width) of the stimulation pulses (which may change any number of times during the stimulation session). Any such characteristics, or any subset of such characteristics, may be referred to as stimulation characteristics.

As a particular example, certain stimulation characteristics may include a current amplitude for one or more current sources coupled to an electrode 126. Such a current source may be an independently-controlled current source that is coupled only to that electrode 126 and not to other electrodes 126 within the electrode array associated with the two or more implanted neurostimulation leads 122. Each such current source may be a “positive” current source that generates a “positive” current directed outward to its corresponding electrode 126 or a “negative” current source that generates a “negative” current directed inward from its corresponding electrode 126, where each electrode 126 has one corresponding “positive” and one corresponding “negative” current source. In certain embodiments, programming module 102 may use the received electrode positioning data 112 to determine appropriate stimulation characteristics to control a number of such current sources to steer stimulation current through targeted nerve tissue in the spinal cord to achieve desired therapeutic results. As an alternative to an independently-controlled current source coupled to a single electrode 126, such a current source may be a current source that is coupled to multiple, or even all, electrodes 126 within the electrode array associated with the two or more implanted neurostimulation leads 122.

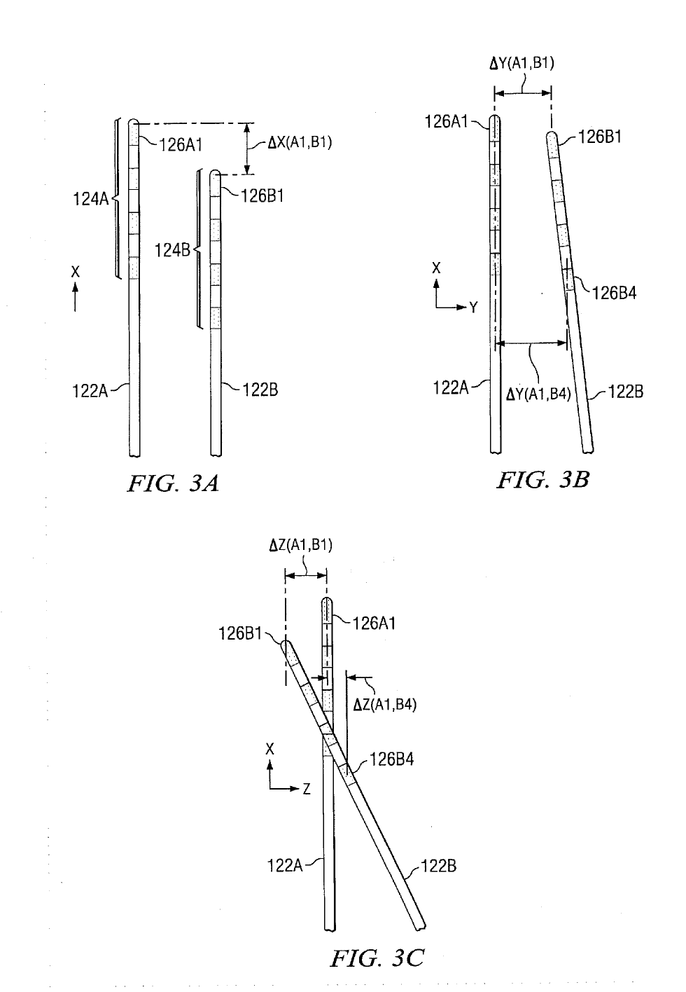

FIG. 2 illustrates two example neurostimulation leads 122 implanted adjacent to nerve tissue in a patient's spinal cord. Although not necessarily to scale, FIG. 2 depicts an example of what a clinician might see using a fluoroscopic imaging system. In the embodiment shown, each lead 122 includes four electrodes 126, although the present contemplates each lead 122 having any suitable number of electrodes 126 according to particular needs. The relative positioning of two or more electrodes 126 may be determined, manually or automatically, and then provided to programming module 102, manually or automatically, for processing.

For example, as shown, electrode 126-A1 of lead 122A may be anatomically superior to electrode 126-B1 of lead 122B. Additionally, as shown, stimulation portion 124A of lead 122A may not be substantially parallel to stimulation portion 124B of lead 122B in a coronal or paracoronal plane (i.e., the projections of leads 122A and 122B onto a coronal or paracoronal plane may not be substantially parallel). Additionally, although not readily visualized in FIG. 2, stimulation portion 124A of lead 122A may also not be substantially parallel to stimulation portion 124B of lead 122B in a sagittal or parasagittal plane (i.e., the projections of leads 122A and 122B onto a sagittal or parasagittal plane may not be substantially parallel). Thus, the distance between a particular electrode 126 of lead 122A and a particular electrode 126 of lead 122B cannot be known merely based on the known design of leads 122A and 122B and associated known spacings between electrodes 126 on each lead 122A and 122B. However, knowledge of this distance may be important for optimum or otherwise satisfactory programming of stimulation characteristics for neurostimulator 120.

Rather than relying on incorrect assumptions to derive an assumed distance between two electrodes 126 on different implanted neurostimulation leads 122, which may lead to suboptimal or unsatisfactory programming of stimulation characteristics, the present invention allows the distance between two electrodes 126 on different implanted neurostimulation leads 122 to be determined to improve the programming of stimulation characteristics. In certain embodiments, relative positioning of two electrodes 126 is determined, manually or automatically, based on the positioning of the electrodes 126 as they appear on one or more fluoroscopic images. In certain other embodiments, relative positioning of two electrodes 126 may be determined, manually or automatically, based on the positioning of the electrodes 126 as revealed using suitable digital imaging technology. For example, relative positioning of two electrodes 126 may be determined from stored digital image data using automated photogrammetry techniques.

For example, a two-dimensional distance between a first electrode 126 in a first lead 122 and a second electrode 126 in a second lead 122 may be determined through direct measurement. If appropriate, this two-dimensional measured distance may be correlated to a suitable coordinate system (e.g., one having an “origin” at a position associated with a particular feature of the first electrode 126 or a particular feature of the patient's anatomy, an “x-axis” extending from the origin in a vertical direction generally parallel to the spinal cord, and a “y-axis” extending in a lateral direction substantially perpendicular to the vertical direction). In a similar manner, a three-dimensional distance between the first electrode 126 in the first lead 122 and the second electrode 126 in the second lead 122 may be measured and, if appropriate, correlated to a suitable coordinate system (e.g., one having an “origin” at a position associated with a particular feature of the first electrode 126 or a particular feature of the patient's anatomy, an “x-axis” extending from the origin in a vertical direction generally parallel to the spinal cord, a “y-axis” extending in a lateral direction substantially perpendicular to the vertical direction, and a “z-axis” extending in an anterior-posterior direction substantially perpendicular to both the vertical and lateral directions).

As another example, the distance between a first electrode 126 in a first lead 122 and a second electrode 126 in a second lead 122 may be measured in a first direction (e.g., a vertical direction generally parallel to the spinal cord) and in a second direction (e.g., a lateral direction substantially perpendicular to the vertical direction). If appropriate, a two-dimensional distance may be calculated based on these two one-dimensional measured distances. If appropriate, as described above, these two one-dimensional measured distances and any calculated two-dimensional distance may be correlated to a suitable coordinate system. In a similar manner, the distance between the first electrode 126 in the first lead 122 and the second electrode 126 in the second lead 122 may be measured in a third direction (e.g., an anterior-posterior direction substantially perpendicular to both the vertical and lateral directions). If appropriate, a three-dimensional distance may be calculated based on these three one-dimensional measured distances. If appropriate, as described above, these three one-dimensional measured distances and any calculated three-dimensional distance may be correlated to a suitable coordinate system.

As another example, two-dimensional coordinates of a first electrode 126 in a first lead 122 and a second electrode 126 in a second lead 122 may be determined, using any suitable coordinate system, and any appropriate one- or two-dimensional distances between the electrodes 126 may be calculated based on the determined coordinates. In a similar manner, three-dimensional coordinates of the first electrode 126 in the first lead 122 and the second electrode 126 in the second lead 122 may be determined, using any suitable coordinate system, and any appropriate one-, two-, or three-dimensional distances between the electrodes 126 may be calculated based on the determined coordinates.

In a particular embodiment, it is assumed that two neurostimulation leads 122 are identical in design, are substantially linear, and are implanted substantially parallel to each other in the same coronal or paracoronal plane. In this case, using any suitable input device coupled to user interface 104, a clinician may input: (1) a value representing a measured distance in a vertical direction (e.g., generally parallel to the spinal cord) between an electrode 126 (e.g., the most cephalad electrode 126) of a first lead 122 and a corresponding electrode 126 (e.g., the most cephalad electrode 126) of a second lead 122; and (2) a value representing a measured distance in a lateral direction (e.g., substantially perpendicular to the vertical direction) between the first lead 122 and the second lead 122. If the leads 122 are instead assumed not to be in the same coronal or paracoronal plane, then in certain embodiments a clinician may also input a value representing a measured distance in an anterior-posterior direction (e.g., substantially perpendicular to the vertical and lateral directions) between the first lead 122 and the second lead 122.

In another particular embodiment, it is assumed that two neurostimulation leads 122 are identical in design, are substantially linear, and are implanted in the same coronal or paracoronal plane but are not substantially parallel to each other. In this case, using any suitable input device coupled to user interface 104, a clinician may input: (1) a value representing a measured distance in a vertical direction (e.g., generally parallel to the spinal cord) between an electrode 126 (e.g., the most cephalad electrode 126) of a first lead 122 and its corresponding electrode 126 (e.g., the most cephalad electrode 126) of a second lead 122; (2) a value representing a measured distance in a lateral direction (e.g., substantially perpendicular to the vertical direction) between an electrode 126 (e.g., the most cephalad electrode 126) of the first lead 122 and its corresponding electrode 126 (e.g., the most cephalad electrode 126) of the second lead 122; and (3) a value representing a measured distance in the lateral direction between a different electrode 126 (e.g., the most caudal electrode 126) of the first lead 122 and its corresponding electrode 126 (e.g., the most caudal electrode 126) of the second lead 122. If the leads 122 are instead assumed not to be in the same coronal or paracoronal plane, then in certain embodiments a clinician may also input a value representing a measured distance in an anterior-posterior direction (e.g., substantially perpendicular to the vertical and lateral directions) between the first lead 122 and the second lead 122.

In another particular embodiment, it is assumed that two neurostimulation leads 122 are identical in design, are substantially linear, are implanted in different coronal or paracoronal planes, and are not substantially parallel to each in any manner (i.e., the projections of leads 122 onto the same coronal or paracoronal plane would be angled relative to each other and the projections of leads 122 onto the same sagittal or parasagittal plane would also be angled relative to each other). In this case, using any suitable input device coupled to user interface 104, a clinician may input: (1) a value representing a measured distance in a vertical direction (e.g., generally parallel to the spinal cord) between an electrode 126 (e.g., the most cephalad electrode 126) of a first lead 122 and its corresponding electrode 126 (e.g., the most cephalad electrode 126) of a second lead 122; (2) a value representing a measured distance in a lateral direction (e.g., substantially perpendicular to the vertical direction) between an electrode 126 (e.g., the most cephalad electrode 126) of the first lead 122 and its corresponding electrode 126 (e.g., the most cephalad electrode 126) of the second lead 122; (3) a value representing a measured distance in the lateral direction between a different electrode 126 (e.g., the most caudad electrode 126) of the first lead 122 and its corresponding electrode 126 (e.g., the most caudad electrode 126) of the second lead 122; (4) a value representing a measured distance in an anterior-posterior direction (e.g., substantially perpendicular to the vertical and lateral directions) between an electrode 126 (e.g., the most cephalad electrode 126) of the first lead 122 and its corresponding electrode 126 (e.g., the most cephalad electrode 126) of the second lead 122; and (5) a value representing a measured distance in the anterior-posterior direction between a different electrode 126 (e.g., the most caudad electrode 126) of the first lead 122 and its corresponding electrode 126 (e.g., the most caudad electrode 126) of the second lead 122.

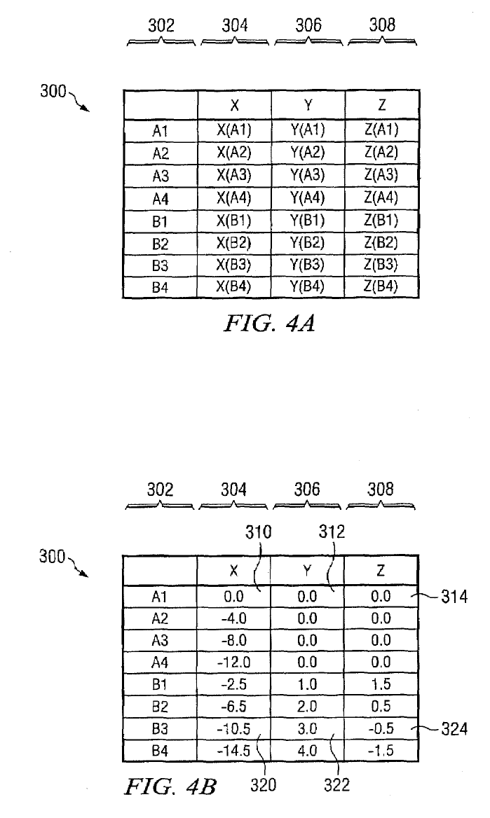

FIGS. 3A-3C illustrate examples of relative positioning of electrodes 126 in example neurostimulation leads 122A and 122B. FIGS. 3A-3C provide examples of the types of relative positioning information that may be determined for one or more implanted electrodes 126. In certain embodiments, distances between electrodes 126 may be determined using one or more of any appropriate features of electrodes 126. For example, a distance between two electrodes 126 may be determined based on the distance between identical features of the electrodes 126, such as their approximate volumetric centers.

Although not necessarily drawn to scale, FIG. 3A illustrates an example view of two implanted neurostimulation leads, 122A and 122B, projected onto a coronal or paracoronal plane. In certain embodiments, a two-dimensional image similar to that illustrated in FIG. 3A may be generated using fluoroscopy, or any other appropriate medical imaging technique. In certain embodiments, prior to measuring the relative positioning of electrodes 126, an axis may be defined relative to one or more features of a neurostimulator lead 122A or 122B and distances determined in the dimension defined by that axis. In the example embodiment shown, an X-axis has been defined substantially parallel to stimulation portion 124A of lead 122A. In relation to this X-axis, a distance ΔX(A1,B1) may be determined as the distance in the direction of the X-axis from electrode 126-A1 to electrode 126-B1, where a positive distance is defined as electrode 126-B1 being above electrode 126-Al within the two-dimensional image (which may be cephalad anatomically to electrode 126-A1). The determined distance ΔX(A1,B1) may be communicated to programming module 102 in any appropriate manner.

Although not necessarily drawn to scale, FIG. 3B illustrates another example view of two implanted neurostimulation leads, 122A and 122B, projected onto a coronal or paracoronal plane. In certain embodiments, prior to measuring the relative positioning of electrodes 126, an axis may be defined in the coronal or paracoronal plane substantially perpendicular to the stimulation portion 124 of a neurostimulation lead 122A or 128B and distances determined in the dimension defined by that axis. In the example embodiment shown, a Y-axis has been defined in the coronal or paracoronal plane substantially perpendicular to stimulation portion of 124A of lead 122A. In relation to this Y-axis, a distance ΔY(A1,B1) may be determined as the distance in the direction of the Y-axis from electrode 126-Al to electrode 126-B1, where a positive distance is defined as electrode 126-B1 being to the right of electrode 126-A1 within the two-dimensional image (which may be to the left anatomically of electrode 126-A1). Similarly, in relation to this Y-axis, a distance ΔY(A1,B4) may be determined as the distance in the direction of the Y-axis from electrode 126-A1 to electrode 126-B4. The determined distances ΔY(A1,B1) and ΔY(A1,B4) may be communicated to programming module 102 in any appropriate manner.

Although not necessarily drawn to scale, FIG. 3C illustrates an example view of two implanted neurostimulation leads, 122A and 122B, projected onto a sagittal or parasagittal plane. In certain embodiments, prior to measuring the relative positioning of electrodes 126, an axis may be defined in the sagittal or parasagittal plane substantially perpendicular to the stimulation portion 124 of a neurostimulation lead 122A or 122B and distances determined in the dimension defined by that axis. In the example embodiment shown, a Z-axis has been defined in the sagittal or parasagittal plane substantially perpendicular to stimulation portion 124A of lead 122A. In relation to this Z-axis, a distance ΔZ(A1,B1) may be determined as the distance in the direction of the Z-axis from electrode 126-A1 to electrode 126-B1, where a positive distance is defined as electrode 126-B1 being to the right of electrode 126-A1 within the two-dimensional image (which may be posterior anatomically to electrode 126-A1). Similarly, in relation to this Z-axis, a distance ΔZ(A1,B4) may be determined as the distance in the direction of the Z-axis from electrode 126-A1 to electrode 126-B4. The determined distances ΔZ(A1,B1) and ΔZ(A1,B4) may be communicated to programming module 102 in any appropriate manner.

In other embodiments, one or more axes and/or dimensions may be based upon features of a medical image. For example, two axes may be defined by the two-dimensions of a fluoroscopy image, with the first axis being directed generally from the bottom to the top of the image, and the second axis being substantially perpendicular to the first axis and directed generally from the left side to the right side of the image.

In other embodiments, one or more axes and/or dimensions may be based upon features of a patient's anatomy. For example, two axes may be defined by the spinal cord in a fluoroscopy image, with the first axis being directed substantially perpendicular to the spinal cord, and the second axis being substantially perpendicular to the first axis in the plane of the image.

In certain embodiments, relative positioning of certain electrodes 126 may be determined by interpolating and/or extrapolating their positions from known or measured positions of other electrodes 126. In certain embodiments, relative positioning of certain electrodes 126 may be determined based on known or measured relative positioning of implanted neurostimulation leads 122 and the known or measured positions of their electrodes 126 relative to certain features of these neurostimulation leads 122. For example, it is possible to obtain additional information pertaining to whether or not a lead lies parallel to the imaging plane. That is, if actual dimension (and the image magnification) is known, then if the imaged distance is shorter, the lead must not lie parallel to the imaging plane (it instead goes partly into or out of the plane). Such information can be used to further improve programming of an implantable pulse generator. For example, positioning of stimulation portion 124A of lead 122A relative to stimulation portion 124B of lead 122B may be determined and then, based on a known spacing of electrodes 126 on neurostimulation leads 122A and 122B, the relative positioning of their electrodes 126 may be determined.

Once the relative positioning of two or more electrodes 126 has been determined, this information may be communicated to programming module 102, with or without user input. For example, this information may be communicated to programming module 102 using one or more peripheral devices, such as a keyboard, mouse, touchpad, or other input device. As a more particular example, a fluoroscopy image may be placed over a touch-screen monitor and the relative positioning of electrodes 126 communicated to programming module 102 by touching each of the relevant electrodes 126 on the touch-screen monitor. As another example, a digital or digitized image may be digitized, and the digitized information may be communicated to programming module 102 and the relative positioning determined automatically. Three-dimensional positioning may be determined using multiple images taken from different angles (e.g., a first image reflecting projections of neurostimulation leads onto a coronal or paracoronal plane and a second image reflecting projections of neurostimulation leads 122 onto a sagittal or parasagittal plane).

In certain embodiments, certain actual or assumed information about the relative positioning of electrodes 126 may be communicated to programming module 102 in a generalized form. For example, a user may communicate to programming module 102 that the stimulation portions 124 of neurostimulator leads 122 are coplanar. As another example, a user may communicate to programming module 102 the particular type of neurostimulation lead 122 or the spacing of electrodes 126 for a particular lead 122.

In certain embodiments, the relative positioning information may be received by programming module 102 and stored in memory module 110 as electrode positioning data 300. FIGS. 4A-4D illustrate example embodiments of electrode positioning data 300. Electrode positioning data 300 may be stored using any suitable data storage format or technique. For example, electrode positioning data may be organized according to the table structure illustrated in FIGS. 4A-4D.

In certain embodiments, as shown in FIG. 4A, electrode positioning data 300 may include three-dimensional rectangular coordinates for each implanted electrode 126. For example, column 302 may contain labels or identifiers for electrodes 126; and columns 304, 306, and 308 may respectively contain the X, Y, and Z rectangular coordinates for each electrode 126, according to any suitable coordinate system. In certain embodiments, the coordinates may be determined relative to a coordinate system with the origin located at a specified anatomical feature, such as the centerline of the spinal cord at the level of a specified vertebrae. In other embodiments, the coordinates may be determined relative to a coordinate system with the origin located at a specified electrode 126. For example, as shown in FIG. 4B, electrode 126-A1 is located at the origin as indicated by zero values in cells 310, 312, and 314. Accordingly, using this example approach, electrode 126-B3 is located at (−10.5 mm, 3.0 mm, −0.5 mm) as shown in cells 320, 322, and 324.

In certain embodiments, as shown in FIG. 4C, electrode positioning data 300 may include a tabular matrix correlating the positioning of each electrode 126 to one or more other electrodes in two or three dimensions. For example, column 302 may contain labels or identifiers for electrodes 126. Column 350 may contain distances in three dimensions between electrode 126-A1 and other electrodes 126. Similarly, columns 352, 354, and 356 may contain distances in three-dimensions between electrodes 126-A2, 126-B1, and 126-B2, respectively, and other electrodes 126. For example, cells 360, 362, and 364 may contain the X, Y, and Z distances between electrodes 126-B2 and 126-B1. For example, as shown in FIG. 4D, electrode 126-A1 is 4 mm from electrode 126-A2 in the x-direction, as indicated by cell 370, and the x-axis is parallel to a line passing through these two electrodes as indicated by zero values in cells 371 and 372.

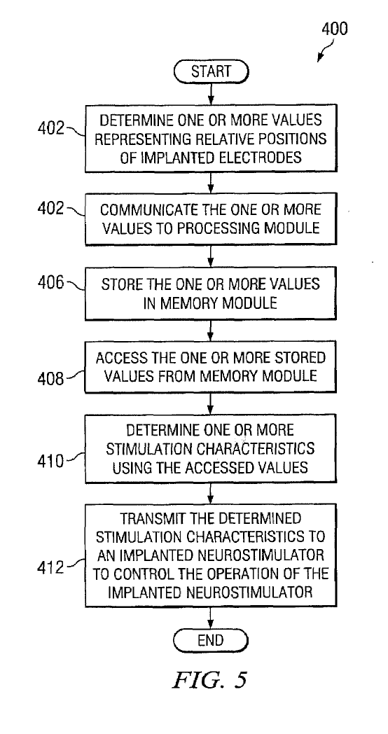

FIG. 5 illustrates an example method 400 for use in programming an implantable neurostimulator 120. At step 402, one or more values representing relative positioning of implanted electrodes 126 are determined. For example, one or more fluoroscopy images of implanted neurostimulation leads 122 may be measured to determine relative positioning values for implanted electrodes 126. At step 404, the one or more values representing relative positioning of implanted electrodes 126 are communicated to programming module 102. For example, the relative positioning values could be communicated to programming module 102 through the use of a peripheral device such as a keyboard, mouse, or touchpad. At step 406, the one or more values representing relative positioning of implanted electrodes 126 are stored in memory module 110. In certain embodiments, the values may be stored according to a table structure illustrated in FIGS. 4A-4D. At step 408, the one or more values representing relative positioning of implanted electrodes 126 are accessed from memory module 110. At step 410, one or more stimulation characteristics are determined using the values accessed from memory module 110. For example, the accessed values may be used to determine appropriate electrodes 126 and/or polarities to utilize to stimulate certain targeted nerve tissue. As another example, the accessed values may be used to determine appropriate parameters, such as amplitude, frequency, and/or pulse-width, for the stimulation pulses. As a particular example, the accessed values may be used to determine a current amplitude for a current source coupled to an electrode 126. At step 412, the determined stimulation characteristics are transmitted to implantable neurostimulator 120 to control the operation of implantable neurostimulator 120. In certain embodiments, for example, the determined stimulation characteristics may be transmitted wirelessly using RF signals.

Thus, method 400 represents a series of steps for programming implantable neurostimulator 120. Method 400 represents an example of one mode of operation, and system 100 contemplates devices using suitable techniques, elements, and applications for performing this mode of operation. Certain steps in the method may take place simultaneously and/or in a different order than shown. In addition, the method may include additional or fewer steps, so long as the method remains appropriate. Moreover, other devices may perform similar techniques to support the programming of neurostimulator 120.

Although certain representative embodiments and advantages have been described in detail, it should be understood that various changes, substitutions and alterations can be made herein without departing from the spirit and scope of the appended claims. Moreover, the scope of the present application is not intended to be limited to the particular embodiments of the process, machine, manufacture, composition of matter, means, methods and steps described in the specification. As one of ordinary skill in the art will readily appreciate when reading the present application, other processes, machines, manufacture, compositions of matter, means, methods, or steps, presently existing or later to be developed that perform substantially the same function or achieve substantially the same result as the described embodiments may be utilized. Accordingly, the appended claims are intended to include within their scope such processes, machines, manufacture, compositions of matter, means, methods, or steps.

1. A computer-implemented system for programming an implantable neurostimulator, comprising:
a memory module operable to store software instructions and relative positioning data representing determined relative positioning in three dimensions of an electrode in a first implanted neurostimulation lead relative to an electrode in a second implanted neurostimulation lead; and
a processing module coupled to the memory module and, according to the software instructions stored in the memory module, operable to:
access the relative positioning data stored in the memory module;
determine one or more stimulation characteristics according to the accessed relative positioning data; and
communicate the one or more stimulation characteristics determined according to the accessed relative positioning data to an implantable neurostimulator, electrically coupled to the first and second implanted stimulation leads, to control operation of the implantable neurostimulator.

2.-20. (canceled)
